(19) United States
(12) Patent Application Publication
(10) Pub. No.: US 2023/0138700 A1
(43) Pub. Date: May 4, 2023

(54) COMBINED ANIMAL-DERIVED AND SYNTHETICALLY PRODUCED PANCREATIC ENZYME REPLACEMENT THERAPY

(71) Applicant: Jai WALL, Marietta, GA (US)

(72) Inventor: Jai WALL, Marietta, GA (US)

(21) Appl. No.: 17/914,914

(22) PCT Filed: Mar. 29, 2021
(86) PCT No.: PCT/US2021/024631

§ 371 Date: Sep. 27, 2022

Related U.S. Application Data
(60) Provisional application No. 63/002,081, filed on Mar. 30, 2020.

(51) Int. Cl. A61K 38/54; A61K 35/39; A61K 38/46; A61K 38/48; A61K 38/47

(52) U.S. Cl. CPC A61K 38/54; A61K 35/39; A61K 38/465; A61K 38/48; A61K 38/47

(57) ABSTRACT

The invention relates to pancreatic enzyme therapy to treat pancreatic enzyme deficiencies. The invention further relates to combining animal-derived pancreatic enzymes with synthetically produced pancreatic enzymes within one delivery vehicle.

### FIELD

This application relates to, among other things, the technical field of pancreatic enzyme therapy and compositions and methods to treat pancreatic enzyme deficiencies such as exocrine pancreatic insufficiency.

### BACKGROUND

Pancreatic enzyme products are used as pancreatic enzyme replacement therapy (PERT) for people with exocrine pancreatic insufficiency (EPI), also known as pancreatic exocrine insufficiency (PEI). Those suffering from EPI may include people with conditions such as cystic fibrosis (CF) or chronic pancreatitis (CP) or those with gluten intolerance, alcoholism, or diabetes or those who have undergone surgical pancreatectomy. Generally PERT medications contain (i) proteases to digest protein, (ii) amylases to digest carbohydrates, and (iii) lipases to digest fat and fat-soluble vitamins. The digestion of these basic food components helps prevent malabsorption, diarrhea, fatty stools, and intestinal infections.

The focus of PERT treatment to date has been to increase the Coefficient of Fat Absorption (CFA) to improve absorption of the fat soluble vitamins, (A, D, E and K) and improve symptoms of steatorrhea. Because the body can produce some amylase and protease from sources other than the pancreas, secondary goals of PERT to date having included improved carbohydrate and protein digestion.

Current Food and Drug Administration (FDA) approved pancreatic enzyme products are animal-derived, specifically from porcine pancreases. Historically, bovine and other animal sources have also been used to generate pancreatic enzyme products. Such animal-derived sources generally contain a mixture of lipase, amylase, and protease. However, the ratio of these enzymes in available animal-sourced products may be unsuitable to effectively treat EPI. For example, higher doses of lipase (rather than amylase or protease) are desired to address the large fat content in the diets of many patients. Also, as mentioned above, the production of amylase and protease production in humans is not solely dependent on the pancreas, making the need to replace amylase and protease less severe as compared to lipase. Yet currently available treatments for PERT such as Creon® have a lipase to amylase to protease ratio of 1:5:3.17 (rounded), which fails to address the more critical need for lipase enzyme replacement in EPI patients. Animal-sourced enzymes such as porcine lipase also have a tendency to break down in acidic environments such as the stomach and duodenum where lipase is needed. This results in lower efficacy of the administered enzymes and the need for higher doses to achieve target CFA levels.

To address these deficiencies in animal-derived pancreatic enzyme products, patients must often take a high number of large-sized pills (e.g., to reach the needed level of lipase to treat their symptoms). In many cases 20-25 capsules or more per day may be required. Pill burden can be particularly daunting for younger patients and those who have difficulty swallowing. To improve CFA for a child with severe CF, for example, six double-0 size capsules would generally be taken with every meal and 3 with every snack. Without PERT the mortality rate in CF is very high but the PERT pill burden reduces compliance.

Synthetic pancreatic enzymes have also been known. Compositions comprising synthetic pancreatic enzymes may contain one type of enzyme, e.g., lipase only, or may contain multiple enzymes. Synthetic enzymes have certain advantages compared to animal-derived pancreatic enzyme products. For example, synthetic pancreatic enzymes are not susceptible to bacterial or viral contaminants in the same way animal sourced enzymes are. Also, as described in U.S. Pat. No. 8,334,130, synthetic lipase with increased resistance to inactivity caused by the low pH of the gastric environment has been known.

Conventionally, pancreatic enzyme products—whether animal derived or synthetically produced—have been used as “single” agents, i.e., capsules of an animal-derived pancreatic enzyme may be used to treat EPI or capsules of a synthetic enzyme are used to treat EPI. Animal and synthetic enzymes have also been used in combination in separate delivery vehicles, e.g., as capsules of synthetic enzyme given with capsules of an animal derived enzyme. However, this just increases the pill burden even further. In either case, optimal digestion, particularly lipid digestion, is not achieved due to difficulties with delivery of the appropriate enzymes at the right time and in the appropriate site (usually in the duodenum). Also, determining and delivering the proper dosage amounts can be difficult and to date the administered dosage of active enzymes tend not to mimic the enzyme profile in amount or type of naturally secreted pancreatic enzymes, particularly in accordance with the age and diet for each individual.

The result is that current EPI treatments require taking multiple, large capsules which are not as effective as they could be and which leads to reduced compliance, inaccurate dosages, and high pill burden.

### SUMMARY OF THE INVENTION

Combining a synthetic pancreatic enzyme such as lipase in the same delivery vehicle with an animal-sourced enzyme or with an enzyme mixture of pancreatic enzymes provides a treatment for all facets of EPI with a lower pill burden. For example if the lipase to amylase to protease ratio in a combination product was 1:2.5:1.58 (doubling the lipase to amylase and protease ratio over an exemplary conventional therapy) the pill burden and/or pill size may be cut in half to maintain the same level of CFA while still supporting carbohydrate and protein digestion. Indeed, animal-derived and synthetically produced pancreatic enzymes may be combined to reduce pill burden to a single delivery vehicle per meal or snack.

The present disclosure provides a composition for pancreatic enzyme replacement therapy (PERT) comprising at least one pancreatic enzyme of animal origin and at least one synthetic pancreatic enzyme in a single delivery vehicle.

In certain embodiments, the above composition may comprise a pH buffering agent. In an embodiment, the pH buffering agent may comprise a bicarbonate and/or a phosphate. For example, the pH buffering agent may comprise sodium bicarbonate, sodium phosphate, or hydrogen phosphate.

In certain embodiments, the composition may comprises a pH adjustment product. In an embodiment, the pH adjustment product may comprise a proton pump inhibitor.

In certain embodiments, the composition may comprises a buffering agent and a pH adjustment product. For example, the composition may comprise a bicarbonate as a buffering agent and a proton pump inhibitor as a pH adjustment product.

In an embodiment, the composition may be for oral, enteral, or parenteral administration.

In an embodiment, the at least one pancreatic enzyme of animal origin may be of mammalian origin. For example, the at least one pancreatic enzyme may be of porcine, bovine, canine, feline, murine, or ovine origin.

In an embodiment, the at least one synthetic pancreatic enzyme may be produced in a yeast host, a bacterium host, a plant cell host, an insect cell host, or an animal cell host.

In an embodiment, the at least one synthetic pancreatic enzyme may be synthesized in vitro.

In an embodiment, the-above mentioned composition may comprise a synthetic lipase, mammalian lipase, mammalian amylase, and mammalian protease.

In an embodiment, the composition may be a solid, liquid, or gel.

In an embodiment, the single delivery vehicle may be selected from a tablet, pill, capsule, sachet, or vial. For example, the single delivery vehicle may comprise a time-controlled tablet, pill, or capsule. In an embodiment, the single delivery vehicle may comprise a delayed-release tablet, pill, or capsule.

In certain embodiments, the composition may comprise lipase, amylase, and protease. For example, in an embodiment, the lipase:amylase:protease ratio may be in the range from 1:10:10 to 10:1:1, preferably from 1:1:1 to 1:6:4.

In a composition as mentioned above, the proportion of synthetic to animal origin pancreatic enzymes may range from 1% to 99%.

The present disclosure also provides a formulation for oral administration in a single delivery vehicle comprising the following enzymes: (i) lipase, (ii) amylase, and (iii) protease, wherein at least one of the enzymes is animal-derived and at least one the enzymes is synthetic.

In certain embodiments, the animal-derived enzyme of such a formulation may be present in a protein-rich fraction derived from an animal extract. For example, the protein-rich fraction of such a formulation may comprise isolated lipase, isolated amylase, and/or isolated protease. In an embodiment such a formulation may comprise animal-derived lipase, animal-derived amylase, and animal-derived protease and the animal-derived enzymes may be present in the same ratio of lipase:amylase:protease as in the animal extract from which the enzymes have been obtained. Alternatively, the formulation may comprise animal-derived lipase, animal-derived amylase, and animal-derived protease and the animal-derived enzymes may be purified or partially purified to increase the proportion of one or more of the enzymes.

In an embodiment, the formulation may comprise synthetic lipase, animal-derived lipase, animal-derived amylase, and animal-derived protease.

The present disclosure also provides a method of treating exocrine pancreatic insufficiency (EPI), comprising: administering via a single delivery vehicle an effective amount of a composition comprising (a) at least one pancreatic enzyme of animal original, and (b) at least one synthetic pancreatic enzyme, to a subject in need thereof.

In an embodiment, such methods include those wherein the EPI is a result of or aggravated by cystic fibrosis (CF), chronic pancreatitis (CP), gluten intolerance, alcoholism, or diabetes.

The present disclosure also provides a method of manufacturing a composition for the treatment of EPI, the method comprising: adding at least one synthetic pancreatic enzyme to at least one pancreatic enzyme of animal origin to form a pancreatic enzyme combination; and filling or forming a single delivery vehicle with the pancreatic enzyme combination.

In an embodiment, such a method may comprise pre-blending at least one pancreatic enzyme of animal origin and at least one synthetic pancreatic enzyme before filling or forming the delivery vehicle.

In an embodiment, such a method may comprise forming the pancreatic enzyme combination at the time of creation of the delivery vehicle.

The present disclosure also provides a single delivery vehicle for oral administration comprising at least one pancreatic enzyme of animal origin and at least one synthetic pancreatic enzyme, which delivery vehicle is configured to maintain physical separation of the at least one pancreatic enzyme of animal origin and the at least one synthetic pancreatic enzyme until ingested.

In an embodiment, the at least one pancreatic enzyme of animal origin and the at least one synthetic pancreatic enzyme may be present in separate chambers within the single delivery vehicle.

In an embodiment, the single delivery vehicle may be configured in a capsule-within-a-capsule format.

In an embodiment, the at least one pancreatic enzyme of animal origin of the single delivery vehicle may be present in a protein-rich fraction derived from an animal extract.

In an embodiment, the protein-rich fraction may be further sub-fractionated to contain a single isolated protein.

Additional features and advantages of the present disclosure will be set forth in part in the description that follows, and in part will be apparent from the description, or may be learned by practice of the present disclosure. The objectives and other advantages of the present disclosure will be realized and attained by means of the elements and combinations particularly pointed out in the description and claims.

The foregoing general description and the following detailed description are exemplary and explanatory only to provide a further explanation of the present disclosure and are not restrictive of the scope of the subject matter encompassed by the claims.

### BRIEF DESCRIPTION OF THE DRAWINGS

FIG. 1 shows a capsule-in-a-capsule configuration for a single delivery vehicle.

FIG. 2A shows a single delivery vehicle comprising microspheres comprising animal-derived pancreatic enzymes (a) and synthetically produced pancreatic enzymes (b). In this embodiment, the microspheres with animal-derived pancreatic enzymes and the microspheres with synthetically produced pancreatic enzymes are interspersed within the capsule such that they are in contact with each other. FIG. 2B shows a single delivery vehicle in which the microspheres comprising animal-derived pancreatic enzymes (a) and synthetically produced pancreatic enzymes (b) are partitioned within the capsule. In this embodiment, microspheres within the capsule comprising animal-derived pancreatic enzymes are not in contact with microspheres comprising synthetically produced pancreatic enzymes.

FIG. 3 shows a tablet comprising animal-derived and synthetically produced pancreatic enzymes, which have been individually produced, combined, and pressed into a tablet having an enteric coating.

### DETAILED DESCRIPTION

The particulars shown herein are by way of example and for purposes of illustrative discussion of the various embodiments of the present disclosure only, and provide what is believed to be the most useful and readily understood description of the principles and conceptual aspects of the disclosed subject matter. In this regard, no attempt is made to show details of the disclosed subject matter in more detail than is necessary for a fundamental understanding of the disclosure, the description making apparent to those skilled in the art how the several forms of the disclosure may be embodied in practice.

The following disclosure refers to more detailed embodiments, with occasional reference to the accompanying figures. The disclosed subject matter, however, may be embodied in different forms and should not be construed as limited to the embodiments set forth herein.

Unless otherwise defined, all technical and scientific terms used herein have the same meaning as commonly understood by one of ordinary skill in the art to which this disclosure belongs. The terminology used in the description herein is for describing particular embodiments only and is not intended to be limiting. As used in the specification and claims, the singular forms “a,” “an,” and “the” are intended to include the plural forms as well, unless the context clearly indicates otherwise. Also, the phrases “at least one” and “one or more” are intended to be interchangeable, and their use is not intended to limit the scope of any described or claimed feature preceded by “a,” “an,” and “the” to a singular form.

All publications, patent applications, patents, and other references mentioned herein are expressly incorporated by reference in their entirety, unless otherwise indicated.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and claims are approximations that may vary depending upon the desired properties sought to be obtained by a particular embodiment. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosed subject matter are approximations, the numerical values set forth in the specific examples are reported as precisely as possible. Any numerical value, however, inherently contains certain errors necessarily resulting from the standard deviation found in the method used to obtain the value. Every numerical range given throughout this specification includes every narrower numerical range that falls within such broader numerical range, as if such narrower numerical ranges were all expressly written herein.

Reference to compounds in the specification includes esters and salts of such compounds. Thus, even if not explicitly disclosed, such esters and salts are contemplated and encompassed by reference to the compounds themselves.

All percent measurements in this application, unless otherwise stated, are measured by weight based upon 100% of a given sample weight. Thus, for example, 30% represents 30 weight parts out of every 100 weight parts of the sample.

The present disclosure relates, in part, to a composition comprising one or more active ingredients, and in certain embodiments, an agent for encapsulating the one or more active ingredients. The composition may be a pharmaceutical composition.

A “pharmaceutical composition” as used herein means a composition comprising an active ingredient and at least one pharmaceutically acceptable excipient. As used herein, the term “pharmaceutically acceptable excipient” means a compound or ingredient that is compatible with the other ingredients in a pharmaceutical formulation and not injurious to an intended subject when administered in normal or therapeutically effective amounts. As used herein, an “intended subject” includes animals and/or humans. The terms “patient” and “subject” may be used interchangeably.

The present disclosure includes a large number and variety of components that are contemplated for inclusion in the pharmaceutical formulations. It should be recognized, however, that where an inventor expressly contemplates including a component, he may also expressly contemplate excluding such component. Thus, with the exception of at least one animal-derived pancreatic enzyme and at least one synthetic pancreatic enzyme, all components disclosed herein should be considered are expressly contemplated for exclusion as well.

As used herein, “active ingredient” is any component of the composition intended to furnish pharmacological activity or other direct effect in the diagnosis, cure, mitigation, treatment, or prevention of disease, or to affect the structure or any function of the body of the intended subject. Active ingredients include those components of the composition that may undergo chemical change during the manufacture of the composition and be present in a finished composition in a modified form intended to furnish the specified activity or effect. Active ingredients also include those components of the finished composition that during or after administration of the finished drug product to the intended user may undergo chemical change to a modified form intended to furnish the specified activity or effect. For example, the active ingredient can be a pharmaceutically acceptable salt of the component that furnishes the specified activity or effect.

Compositions or formulations as described herein may contain one type of active ingredient (for example, an animal-derived lipase and a synthetic lipase), or more than one active ingredient, such as two, three, four, five, six, seven, eight, or nine active ingredients, or more than nine active ingredients.

Compositions for pancreatic enzyme replacement therapy (PERT) comprising at least one pancreatic enzyme of animal origin and at least one synthetically produced pancreatic enzyme in a single delivery vehicle are specifically contemplated. As mentioned above, combining a synthetic pancreatic enzyme such as lipase in the same delivery vehicle with an animal-sourced enzyme mixture of pancreatic enzymes (such as lipase, amylase, and protease) is capable of providing a treatment for EPI with a lower pill burden. For example, as contemplated herein, a single delivery vehicle comprising a composition with at least one pancreatic enzyme of animal origin and at least one synthetically produced pancreatic enzyme may be administered with food or drink (e.g., per meal or per snack or per drink or per shake) to effectively treat EPI.

Enzymes

The types of digestive and/or pancreatic enzymes contemplated for use herein include lipases, amylases, and proteases as well as other enzymes and/or proteins secreted by the pancreas, salivary glands, etc., which may be synthetically produced or obtained from natural sources, and/or which have the ability to break down fats, proteins, and/or starch. While the pancreas produces additional enzymes, including digestive enzymes such as ribonuclease, deoxyribonuclease, gelatinase, and elastase, such additional digestive and/or metabolic enzymes are not associated with current PERT regimes.

As used herein “lipase” refers to an enzyme that catalyzes the breakdown of fats to fatty acids and glycerol or other alcohols. Non-limiting examples of lipases as contemplated herein include colipase, procolipase, phospholipase A2, cholesterol esterase, pancreatic lipase related protein 2 (PLRP 2), etc. As contemplated herein, the lipase may include be gastric lipase, pancreatic lipase, hepatic lipase, or salivary lipase. In an embodiment, pancreatic lipases are preferred.

As used herein “amylase” refers to an enzyme that converts starch and glycogen into simple sugars. Non-limiting examples of amylases as contemplated herein include α-amylases, β-amylases, γ-amylases, acid α-glucosidases, salivary amylases such as ptyalin, etc.

As used herein “protease” refers to an enzyme that catalyzes the breakdown of proteins and/or peptides. Proteases include proteinases, peptidases, or proteolytic enzymes. Proteases also include aspartic acid peptidase, cysteine peptidase, metallopeptidases, serine peptidases, threonine peptidases, etc. Non-limiting examples of proteases include trypsin, chymotrypsin, carboxypeptidase A carboxypeptidase B, elastase, and kininogenase.

Animal-Derived Pancreatic Proteins

As used herein, an “animal-derived pancreatic protein” or “animal-derived pancreatic enzyme” refers to a natural or wild-type protein that has been extracted, concentrated, purified, or isolated (either partially or completely) from the pancreas or pancreatic secretion of an animal. As referred to herein, “animal-derived,” “animal-sourced,” and “of animal origin” may be used interchangeably. The animal is not particularly limited and may include a mammal, amphibian, reptile, or bird. For example, the animal-derived pancreatic protein may obtained from the pancreas of a mammal such as a pig, cow, mouse, cat, porpoise, whale, dolphin, sheep, monkey, or dog. In an embodiment, the animal-derived pancreatic protein may be a mixture of enzymes obtained from a pancreatic secretion of an animal and may include lipase, amylase, and protease (e.g., pancreatin). As used herein, an “animal-derived pancreatic protein” or “animal-derived pancreatic enzyme” does not include pancreatic enzymes produced by recombinant DNA technology in a cell or cell line derived from an animal.

Synthetic Pancreatic Proteins

As used herein, a “synthetic pancreatic protein” or “synthetic pancreatic enzyme” is a pancreatic protein produced by recombinant DNA technology by a suitable host cell or a chemically modified or produced pancreatic protein. As referred to herein, “synthetic” and “synthetically produced” may be used interchangeably. Non-limiting examples of a synthetic pancreatic protein include those produced via recombinant DNA technology such as by expression of an exogenous DNA sequence which has been introduced into a host cell such as bacterial, yeast, fungal, plant, insect, or mammalian host cell. Alternatively, a synthetically produced protein as contemplated herein may be produced artificially in vitro and/or via chemical synthesis.

Other “Pancreatic” Proteins

In an embodiment, the “pancreatic” enzymes contemplated herein may be extracted, concentrated, purified, or isolated (either partially or completely) from an organism other than an animal such as a bacterium, fungus or plant. For example, the use of bacterial-derived amylases, fungal-derived proteases, and plant-derived lipases (or any combination thereof) may also be included in the compositions or formulations described herein. Examples of such proteins include natural or wild-type bacterial proteins (e.g., bacterial lipase, bacterial amylase, bacterial protease), natural or wild-type fungal proteins (e.g., fungal lipases, fungal amylase, and fungal protease), and natural or wild-type plant proteins (e.g., plant lipase, plant amylase, and plant protease). In an embodiment, microbial lipases of fungal origin may be included in the compositions or formulations described herein. In certain embodiments, such enzymes may have higher stability and/or activity in acidic environments such as the stomach and duodenum. In certain embodiments such proteins may be able to function without bile or colipase. In an embodiment, the lipase may be from a fungus such as Aspergillus niger, Aspergillus melleus, Thermomyces lanuginosa, Yarrowia lipolytica, etc., or from a bacteria such as a Psuedomonas sp., Burkholderia sp., etc.

Combining Animal-Derived and Synthetic Proteins

The types of animal-derived and synthetic pancreatic proteins that may be combined and the manner in which the various proteins are combined is not particularly limited. For example, synthetic lipase may be added to an animal-derived mixture of lipase, protease, and amylase. Alternatively, two or three different synthetic pancreatic proteins may be produced separately, pre-blended, and then combined with one isolated animal-derived protein. The various pancreatic enzymes contemplated herein (including protease, amylase, and lipase, whether sourced from an animal, plant, fungus, or bacteria, or produced via chemical or recombinant technology) and any mixture thereof, should be considered as combinable with each other as single preparations, paired enzymes, or mixtures of three or more enzymes. Options and exemplary configurations for such compositions and formulations and single delivery vehicles comprising such compositions and formulations are described as follows.

Ratios and Proportions

The ratios of lipase:amylase:protease in the compositions, formulations, or delivery vehicles (for the synthetic, animal-derived, or combination of enzymes) may be in the range of about 1:10:10 to about 10:1:1. In an embodiment, the ratio may be in the range of about 1:6:6: to about 6:1:1 or about 1:5:5 to about 5:1:1 or about 1:6:4 to about 1:1:1. Other exemplary ratios include 1:5:3.17 to 1:2.5:1.58 to 1:0.50:0.32 and ratios in between and 1.0:1.0:0.15 to about 1.0:1.0:1.15. Other possible ratios and/or ranges of ratios for lipase:amylase:protease include 1:4.2-4.8:3.13-3.4 or 1:3.78:3.59 or 1:4-5.86:2.38-3.38. Ratios may be based on weight (e.g., mg), mass, or enzyme activity. Where not explicitly stated, ratios not expressed as a percentage should be understood herein to be based on activity, i.e., in International Units (IU) or the amount which will catalyze the transformation of one micromole of substrate per minute under standard conditions. Alternatively, activity may be expressed in United States Pharmacopeia (USP) units.

The proportion of synthetic to animal-sourced pancreatic enzymes may range from 1% to 99% by weight (e.g., from 2% to 98%, 3% to 97%, 4% to 96%, 5% to 95%, 6% to 94%, 7% to 93%, 8% to 92%, 9% to 91%, 10% to 90%, 15% to 85%, 20% to 80%, 25% to 75%, 30% to 70%, 40% to 60%, or about half and half (50%).

The compositions, formulations, and delivery vehicles described herein may comprise animal-derived lipase, animal-derived amylase, and animal-derived protease in the same ratio of lipase:amylase:protease as in the animal extract from which the enzymes have been obtained. Alternatively, the compositions, formulations, and delivery vehicles may comprise animal-derived enzymes that have been concentrated, purified or partially purified to increase the proportion of one or more of the enzymes.

Activity

Lipase activities in the compositions, formulations, or delivery vehicles as described herein may be in the range of about 4500-150,000 IU, for example about 5,500-145,000 IU, about 7,500-125,000 IU, about 8,500-100,000 IU, about 9,500-95,000 IU, about 11,500-85,000 IU. In certain embodiments the lipase activity may be about 5,000 IU, 6,000 IU, 7,000 IU, 8,000 IU, 9,000 IU, 10,000 IU, 11,000 IU, 12,000 IU, 13,000 IU, 14,000 IU, 15,000 IU, 16,000 IU, 17,000 IU, 18,000 IU, 19,000 IU, 20,000 IU, 25,000 IU, 30,000 IU, 40,000 IU, 50,000 IU, 60,000 IU, 70,000 IU, 80,000 IU, 90,000 IU, 100,000 IU, 110,000 IU, 120,000 IU, 130,000 IU, or 140,000 IU or more.

Amylase activities in the compositions, formulations, or delivery vehicles as described herein may be about 4,500-150,000 IU, for example about 5,000-125,000 IU, about 10,000-100,000 IU, about 15,000-90,000 IU, about 20,000-80,000 IU, about 25,000-75,000 IU, or about 30,000-60,000 IU. In certain embodiments the amylase activity may be about 4,500-5,500 IU, about 9,000-11,000 IU, about 13,500-16,500 IU, and about 18,000-22,000 IU. In certain embodiments the amylase activity may be less than that of the lipase activity. In certain embodiments, the amylase activity may be 50,000 IU, 40,000 IU, 30,000 IU, 25,000 IU, 20,000 IU, 19,000 IU, 18,000 IU, 17,000 IU, 16,000 IU, 15,000 IU, 14,000 IU, 13,000 IU, 12,000 IU, 11,000 IU, 10,000 IU, 9,000 IU, 8,000 IU, 7,000 IU, 6,000 IU, or 5,000 IU or less.

Protease activities in the compositions, formulations, or delivery vehicles as described herein may be about 4,500-150,000 IU, for example about 5,000-125,000 IU, about 8,000-34,000 IU, about 17,000-67,000 IU, about 26,000-100,000 IU, about 35,000-134,000 IU, about 10,000-100,000 IU, about 20,000-80,000 IU, about 25,000-75,000 IU, or about 30,000-60,000 IU. In certain embodiments the protease activity may be less than that of the lipase activity. In certain embodiments, the protease activity may be 50,000 IU, 40,000 IU, 30,000 IU, 25,000 IU, 20,000 IU, 19,000 IU, 18,000 IU, 17,000 IU, 16,000 IU, 15,000 IU, 14,000 IU, 13,000 IU, 12,000 IU, 11,000 IU, 10,000 IU, 9,000 IU, 8,000 IU, 7,000 IU, 6,000 IU, or 5,000 IU or less.

The total amount of animal-derived and synthetically produced pancreatic enzymes in the compositions, formulations, and delivery vehicles described herein (by weight) may range from about 20-100%, 20-90%, 20-80%, 20-70%, 20-60%, 20-50%, 20-40%, 20-30%, or about 20%, about 30%, about 40%, about 50%, about 60%, about 70%, about 80%, about 90%, 91%, 92%, 93%, 94%, 95%, 96%, 97%, 98%, 99%, or about 100%. In one embodiment, the total amount of animal-derived and synthetically produced pancreatic enzymes is 65-96%. In another embodiment, the total amount of lipase, amylase, and protease is about 75-85%.

While the ratios, proportions, and activities may be selected to address patient CFA, these parameters are not particularly limiting and may be selected to achieve target coefficient of nitrogen absorption (CNA), stool fat excretion (SFE), stool nitrogen excretion (SNE), stool weight (SW) levels.

pH Buffering Agents

Pancreatic enzymes are most active at neutral or slightly alkaline conditions.

Accordingly, the delivery vehicle may contain a pH buffer or a pH adjusting product.

The compositions, formulations, and delivery vehicles contemplated herein may comprise a pH buffering agent in addition to the animal-derived and synthetic pancreatic enzymes. For example, the pH buffering agent may comprises a bicarbonate and/or a phosphate. In an embodiment, the pH buffering agent comprises sodium bicarbonate, sodium phosphate, hydrogen phosphate, acetic acid, citric acid, histidine, and/or tromethamine, etc. In an embodiment, the pH buffering agent may be used in combination with one or more pH adjustment products.

pH Adjustment Products

The compositions, formulations, and delivery vehicles contemplated herein may comprise a pH adjustment product in addition to the animal-derived and synthetic pancreatic enzymes. In an embodiment, the pH adjustment product may comprise a proton pump inhibitor, an antacid, a potassium competitive acid blocker or an 112 receptor antagonist. Non-limiting examples of proton pump inhibitors include omeprazole, lansoprazole, dexlansoprazole, rabeprazole, esomeprazole, and esomeprazole magnesium, etc. Non-limiting examples of antacids include bismuth subsalicylate and simethicone. Non-limiting examples of 112 (histamine) receptor antagonist include ranitidine, famotidine, and cimetidine.

In an embodiment, the composition may comprise a bicarbonate as a buffering agent and a proton pump inhibitor as a pH adjustment product.

Coatings and Time-Controlled Release

It is often advantageous to deliver the pancreatic enzymes to the duodenum. Accordingly, in an embodiment, enteric coated dosage forms are contemplated. The pancreatic enzymes may be present in a coated particle comprising a core and a coating. The coating may be an enteric coating. For example, capsules may comprise hydroxymethyl cellulose or a polymer such as PEG, polyoxyethylene, copolymers of polyoxyethylene-polyoxypropylene, etc. as described in U.S. Pat. No. 9,259,393.

Enteric coatings can protect the pancreatic enzymes and enzyme mixtures described herein from degradation as they move through the acid environment of the gut (e.g., stomach) and to effectively release the pancreatic enzymes in the duodenum with chyme. In certain embodiments, the enteric coatings may be designed to dissolve above pH 5 so that contents are released in the duodenum. Enteric coatings are known in the art. Non-limiting examples of polymers which may be applied in this regard include hydroxypropylmethylcellulose phthalate, hydroxypropylmethylcellulose acetate succinate, acrylates, and polyvinylacetophthalate. Plasticizing agents such as triethylcitrate and dibutylphthalate may also be added as well as a gliding agent such as talc to reduce the adhesion among the microspheres during coating. In an embodiment, the enteric coating may comprise agar, acrylic acid-based polymers, carboxymethyl cellulose, carboxymethylethyl cellulose, carrageen, cellulose acetate phthalate, chitin, corn protein extract, ethyl cellulose, gum arabic, hydroxypropyl cellulose, methacrylic acid-ethyl methacrylate-copolymer, methyl cellulose, polyvinyl alcohol, sodium alginate, and/or starch acetate phthalate, etc.

In certain embodiments, the single delivery vehicle may be configured for time-controlled delivery, i.e., as a delayed-release composition or a non-delayed release composition. Alternatively, the single delivery vehicle may be configured for immediate release upon administration.

Microspheres, Minimicrospheres, and Microtabs

In certain embodiments, the pancreatic enzymes as described herein may be comprised in microspheres and/or minimicrospheres. The microspheres and/or minimicrospheres may be further combined, for example, into a single delivery vehicle. For example, microspheres having the pancreatic enzymes may have a diameter between 10 μm and 1500 μm, between 20 μm and 1300 μm, between 30 μm and 1100 μm, between 40 μm and 1300 μm, between 50 μm and 1000 μm, or between 100 μm and 800 μm. In an embodiment, minimicrospheres may comprise gastro-resistant granules and may be combined to obtain specific amounts and/or activities of enzyme, (e.g., 5000-25000 IU lipase, 5100-30000 IU amylase, and/or 320-19000 IU protease). In addition to the pancreatic enzymes, the microspheres or minimicropheres may comprises one or more hydrophilic low-melting polymers and excipients. Examples of hydrophilic low-melting polymers include polyethylene glycol, polyoxyethylene, and copolymers of polyoxyethylene-polyoxypropylene, etc.

Microtabs may be in the form of small coated tablets and their method of administration is not particularly limited. Like microspheres and minimicrospheres, microtabs may be combined into a single delivery vehicle.

The pancreatic enzyme content of the microspheres (by weight) may range from about 20-100%, 30-90%, 40-80%, 40-70%, 40-60%, 40-50%, or about 20%, about 30%, about 40%, about 50%, about 60%, about 70%, about 80%, about 90%, or greater.

In certain embodiments, microspheres comprising the pancreatic enzymes may be formed to more evenly and rapidly disperse the enzymes in chyme. Spheronization is also a relatively quick and inexpensive process which does not require the use of organic solvents or water and may be performed at temperatures higher than the denaturation temperature of the pancreatic enzymes to be spheronized. Microspheres also provide other advantages as well. For example, a reduced quantity of coating agent is needed than for irregularly shaped compositions, and the process times are reduced compared to other compositions, thereby lowering costs and mitigating risk of enzyme inactivation.

In an embodiment, the microspheres, minimicrospheres, or microtabs themselves may be individually coated with one or more coatings as described herein. For example, a single delivery vehicle may comprise animal-derived enzymes present in coated microspheres, synthetic enzymes present in coated microspheres, both animal-derived and synthetic enzymes present in coated (or uncoated) microspheres, or any combination of microspheres which are coated/uncoated and which contain animal-derived/synthetic enzymes. In a preferred embodiment, the single delivery vehicle may comprise both animal-derived and synthetic enzymes present in separate, individually coated microspheres. The one or more coatings may provide certain advantages. For example, enteric coatings may protect the pancreatic enzymes and enzyme mixtures described herein from degradation as they move through the acid environment of the gut. Enteric coatings may also protect the contents of the microspheres individually during preparation, storage, transport, and other handling, including from possible degradation from enzymes or other substances present in the same delivery vehicle and/or from enzymes or other substances present in other adjacent or proximal microspheres.

Powders, Granules, Emulsions, Suspensions, Nanoparticles

In certain embodiments the pancreatic enzymes may be formulated for manufacturing purposes or delivery in the form of powders (wet or dry), granules, suspensions, emulsions, and/or nanoparticles. Nanoparticles may take the form of lipid nanoparticles, liposomes, micelles, polymer particles, inorganic particles, hydrogels, nanoemulsions, nanosuspensions, and the like. For example, mesoporous nanoparticles such as porous silicon, porous silica, and halloysite may provide for co-loading of both the animal-derived and synthetic pancreatic enzymes. The mesoporous nanoparticles may be encapsulated in pH-controlled polymers to deliver the pancreatic enzymes to the gut.

Excipients

Suitable excipients are known to those of skill in the art and examples are described, for example, in the Handbook of Pharmaceutical Excipients (Kibbe (ed.), 3rd Edition (2000), American Pharmaceutical Association, Washington, D.C.), and Remington's Pharmaceutical Sciences (Gennaro (ed.), 20th edition (2000), Mack Publishing, Inc., Easton, Pa.), which, for their disclosures relating to excipients and dosage forms, are incorporated herein by reference. Examples of excipients include but are not limited to fillers, extenders, diluents, wetting agents, solvents, emulsifiers, preservatives, absorption enhancers, sustained-release matrices, starches, sugars, microcrystalline cellulose, granulating agents, lubricants, binders, disintegrating agents, coloring agents, release agents, coating agents, sweetening agents, flavoring agents, perfuming agents, antioxidants, plasticizers, gelling agents, thickeners, hardeners, setting agents, suspending agents, surfactants, humectants, carriers, stabilizers, and combinations thereof.

Non-limiting examples of suitable excipients acetyl alcohol, dimethicone, hypromellose phthalate, polyethylene glycol, triethyl citrate, colloidal silicon dioxide, crosscarmellose sodium, lactose monohydrate, microcrystalline cellulose, stearic acid, and talc, etc.

Stability

In some embodiments, the compositions of the invention will be stable at room temperature. As used herein, “stable” means capable of storage and deliverable to the duodenum without significant alteration in the fundamental composition such that the active ingredients remain usable as intended. In some embodiments, the compositions will be stable at room temperature; in some embodiments, the compositions will be stable under reduced temperature conditions, such as refrigeration or freezing. In some embodiments, the compositions are stable for one or more days, such as a week or month or more. In some embodiments, the compositions will include one or more additional ingredients to improve stability, such as antioxidants.

Delivery Vehicles

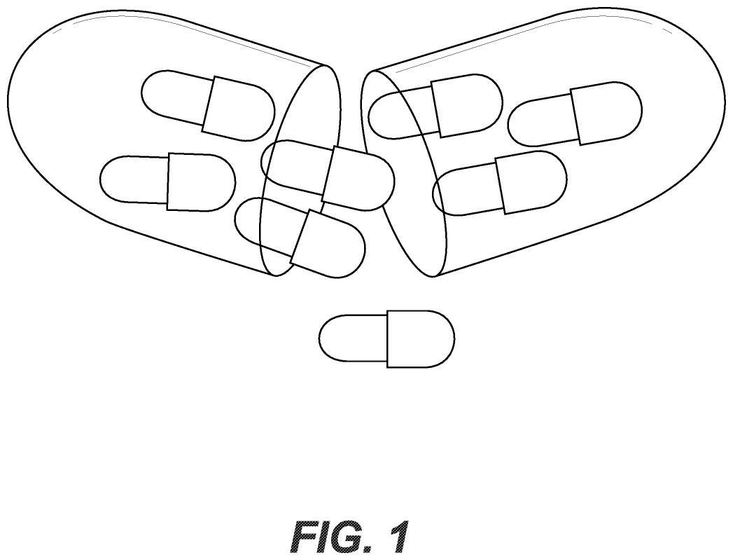

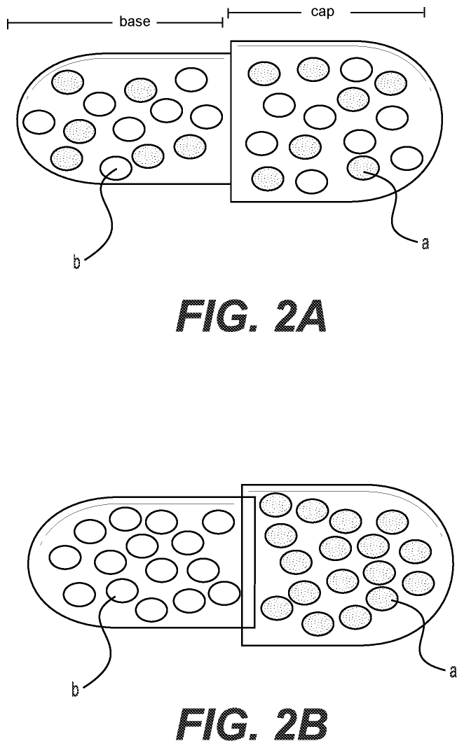

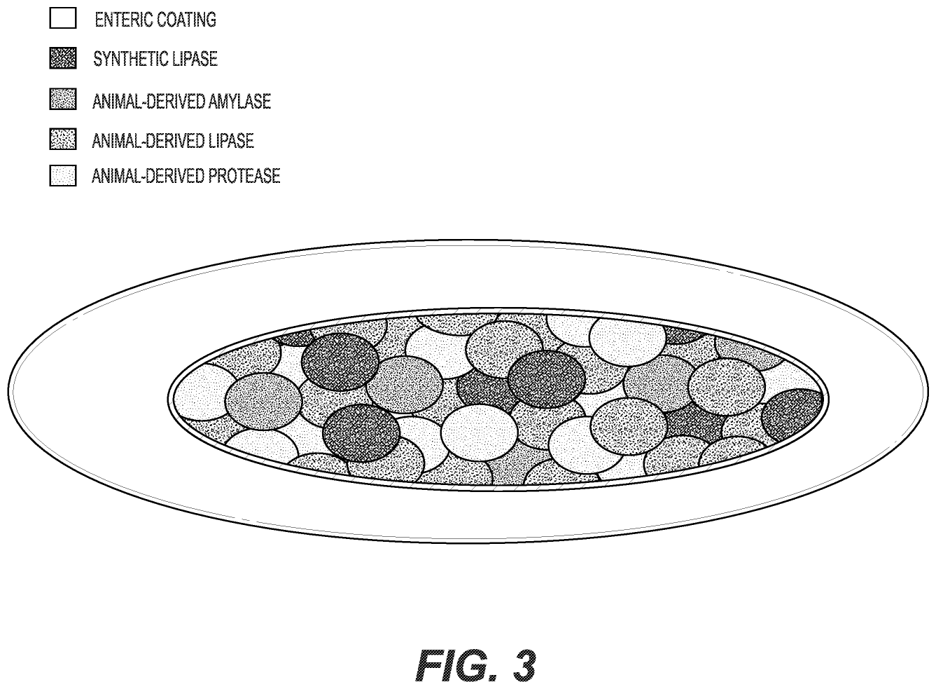

Delivery vehicles as contemplated include single delivery vehicles for oral administration. In an embodiment, the single delivery vehicle comprises one or more pancreatic enzymes of animal origin and at least one synthetic pancreatic enzyme and may be configured to maintain physical separation of the one or more pancreatic enzymes of animal origin and the at least one synthetic pancreatic enzyme until ingested. In an embodiment, the one or more pancreatic enzymes of animal origin and the at least one synthetic pancreatic enzyme may be present in separate chambers within the single delivery vehicle. For example, the single delivery vehicle may be configured in a capsule-within-a-capsule format (e.g., a larger capsule which forms a disintegrable housing for smaller, also disintegrable capsules that are present inside the larger capsule; see FIG. 1). In certain alternative embodiments, the animal-derived pancreatic enzyme(s) may be present in a first microsphere (or minimicrosphere or microtab), and the synthetically produced pancreatic enzyme(s) may be present in a second microsphere (or minimicrosphere or minitab) and the microspheres (or minimicrospheres or minitabs) may be combined within a single capsule (FIG. 2A and FIG. 2B show microsphere embodiments). In an embodiment, the one or more pancreatic enzymes of animal origin (a) may be present in the cap of a capsule and the one or more synthetically produced pancreatic enzymes may be present in the body of a capsule (b) which has been partitioned from the cap as shown in FIG. 2B. In yet another embodiment, animal-derived pancreatic enzymes and a synthetically produced pancreatic enzyme may be produced separately, then combined, and pressed into tablet form (FIG. 3).

The size of the capsule or tablet may be selected from those standard in the art, may vary, and are not particularly limited. For example, capsules ranging from size 000 (holding approximately 615-1370 mg) to 5 (holding approximately 60-130 mg) are contemplated, although capsule sizes of 0 (holding approximately 305-680 mg) and 1 (holding approximately 225-500 mg) are preferred. Capsule sizes 00 (holding approximately 430-950 mg), 2 (holding approximately 165-370 mg), 3 (holding approximately 135-300 mg), and 4 (holding approximately 95-210 mg) are also contemplated. The length of the delivery vehicle (e.g., the locked length of a capsule) may range from 11-27 mm, for example, from 11.10-26.14 mm, from 14.3 to 23.3 mm, from 15.9 to 21.7 mm, or from 18.0-19.4 mm. The cap diameter may range from 4.91-9.91 mm and the body diameter may range from 4.68-9.55 mm in a manner determined mathematically to correspond to any of the lengths contemplated herein and/or to achieve a volume of 1.37-0.13 ml, 0.21-0.95 ml, 0.30-0.68 ml, or 0.37 to 0.50 ml.

Delivery vehicles as described herein also include capsules, pellets, pills, tablets, sachets, and vials.

Treatment Methods

Methods of treating exocrine pancreatic insufficiency (EPI) and other pancreatic enzyme deficiencies are all contemplated. Treatment methods may include administering via a single delivery vehicle an effective amount of a composition comprising (a) at least one pancreatic enzyme of animal origin, and (b) at least one synthetic pancreatic enzyme, to a subject in need thereof. In certain embodiments, the EPI may be a result of (or aggravated by) cystic fibrosis (CF), chronic pancreatitis (CP), gluten intolerance, alcoholism, diabetes, pancreatic cancer, Shwachman-Diamond Syndrome, and/or inflammatory bowel disease, etc. By combining at least one pancreatic enzyme of animal origin and at least one synthetically produced pancreatic enzyme, the ratio of lipase:amylase:protease may be tailored to a patient's needs or to a patient's diet or digestive profile, thereby ensuring more individualized and effective treatment. Also, by combining animal-derived and synthetic proteins as contemplated herein, the specific activity of the pancreatic enzymes may be increased and volume size of the composition may be reduced, thereby lowering pill burden, particularly for younger patients and patients with difficulty swallowing.

The compositions, formulations, and delivery vehicles may be administered as described herein may be administered orally, enterally, or parenterally. In a preferred embodiment, the compositions, formulations, and delivery vehicles are administered orally.

The amount of the dose of the active ingredient administered, as well as the dose frequency, will vary depending on the particular dosage form used and route of administration. The amount and frequency of administration will also vary according to the age, body weight, and response of the individual subject or patient. However, as contemplated herein a single delivery vehicle will typically be administered with each meal or snack.

Manufacturing Methods

Methods of manufacturing compositions, formulations, and delivery vehicles as described herein are not particularly limited. For example, in an embodiment the methods for manufacturing a composition for the treatment of EPI as contemplated herein comprises adding at least one synthetic pancreatic enzyme to at least one pancreatic enzyme of animal origin to form a pancreatic enzyme combination; and filling or forming a single delivery vehicle with the pancreatic enzyme combination.

The timing and/or order in which the contents are added is not particularly limited. In an embodiment, the method may comprise pre-blending at least one pancreatic enzyme of animal origin and at least one synthetic pancreatic enzyme before filling or forming the delivery vehicle. In an embodiment, the method comprises forming the pancreatic enzyme combination at the time of creation of the delivery vehicle.

In the case of a single delivery vehicle comprising microspheres, the microspheres may be obtained by mixing solid pancreatic enzymes with one more hydrophilic low-melting copolymers and heating to a temperature greater than the melting temper of the polymer under stirring conditions as disclosed in U.S. Pat. No. 9,259,393. Microspheres may help to reduce pill size and provide high/stable enzymatic activity.

Manufacturing Methods for Animal-Derived Pancreatic Enzymes

Pancreatic enzymes of animal origin may be concentrated, isolated, purified, or extracted via methods known to those of skill in the art. For example, the pancreatic enzymes of animal origin may be first obtained in a pancreatic extract. The protein content of the pancreatic extract may then be enriched by fractionation (e.g., eluted from a column or columns) so that a protein-rich fraction derived from an animal extract is obtained. The protein-rich fraction may optionally be further sub-fractionated to contain ever purer or more active protein fractions or to obtain single isolated protein (e.g., lipase).

In an embodiment, viral and microbial contaminants are removed from the composition. Viral and microbial contaminants may be removed via conventional processes know to those of skill in the art such as electron beam irradiation, filtration, etc.

Manufacturing Methods for Synthetic Pancreatic Enzymes

In an embodiment, the at least one synthetic pancreatic enzyme may be synthesized in vitro.

In another embodiment, the synthetic pancreatic enzymes are produced via expression from a transgene introduced into a suitable host cell. One or more (e.g., several) of the enzymes in the enzyme composition may be wild-type proteins expressed by the host strain, recombinant proteins, or a combination of wild-type proteins expressed by the host strain and recombinant proteins. For example, one or more (e.g., several) enzymes may be native proteins of a cell, which is used as a host cell to express recombinantly the enzyme composition.

Suitable bacterial host cells include Escherichia coli, Pseudoalteromonas haloplanktis, Shewanella sp. strain Ac10, Pseudomonas fluorescens, Pseudomonas putida, Pseudomonas aeruginosa, Halomonas elongata, Streptomyces lividans, Streptomyces griseus, Mycobacterium smegmatis, Clostridium spp. such as Clostridium perfringens, Corynebacterium glutamicum, Corynebacterium ammoniagenes, Brevibacterium lactofermentum, Bacillus subtilis, Bacillus brevis, Bacillus megaterium, Bacillus licheniformis, Bacillus amyloliquefaciens, Lactobacillus gasseri, Lactobacillus reuteri, Lactobacillus casei, Lactobacillus plantarum, and Lactobacillus lactis, Burkholderia pseudomallei, Burkholderia cepacia, and Burkholderia glumae.

Suitable yeast or fungal hosts include Saccharomyces cerevisiae, Candida albicans, Pichia pastoris, and Cryptococcus neoformans and species of Aspergillus, Trichoderma, Penicillium, and Rhizopus, including for example, Aspergillus niger, etc.

Suitable plants and plant cells for production of pancreatic enzymes include Arabidopsis thaliana, Nicotiana tabacum BY2, Nicotiana benthamiana, Daucus carota, Carica papaya, Physcomitrella patens, Lemna sp., Spirodela sp. etc.

Suitable insect cells for production of pancreatic enzymes include, but are not limited to, Drosophila S2, Spodeptera frugiperda cells, Sf9 and Sf21 cells.

Suitable mammalian cell lines or cell culture host cells include human cell lines, monkey cell lines, mouse cells lines, etc. For example, potential cells lines contemplated for such use herein include HEK293 cells, HT-1080 cells, COS7 cells, PER.C6 cells, CHO cells, HeLa cells, and BHK-21 cells

### EXAMPLES

### Example 1

A size 0 capsule will be manufactured having synthetically produced lipase and animal-derived lipase, amylase, and protease in a final lipase:amylase:protease ratio of 1:2.1:1.58. The animal-derived enzymes will be isolated and concentrated from porcine pancreatic extract, whereas the synthetic lipase will be recombinantly produced in vitro via expression in a recombinant human host cell line. The animal-derived and synthetic proteins will then be pre-blended, granulated, and loaded into capsules. Each capsule will represent a significant reduction in pill burden compared to a conventionally sized PERT delivery dose (multiple size 00 pills) each containing a lipase:amylase:protease ratio of 1:4.2:3.15.

### Example 2

A size 1 capsule will be manufactured in a manner substantially similar to Example 1. The size 1 capsule will have synthetically produced lipase and animal-derived amylase and protease in a final lipase:amylase:protease ratio of 1:2.1:1.58.

### Example 3

A size 00 capsule will be manufactured having synthetically produced lipase and animal-derived lipase, amylase, and protease in a final lipase:amylase:protease ratio of 1:0.35:0.26. Such a capsule will represent a significantly more effective single dosage compared to a conventionally sized PERT delivery vehicle (size 00 pill) containing a lipase:amylase:protease ratio of 1:4.2:3.15.

What is claimed is:

1. A composition for pancreatic enzyme replacement therapy (PERT) comprising at least one pancreatic enzyme of animal origin and at least one synthetic pancreatic enzyme in a single delivery vehicle.

2. The composition according to claim 1, which further comprises a pH buffering agent.

3. The composition according to claim 2, wherein the pH buffering agent comprises a bicarbonate and/or a phosphate.

4. The composition according to claim 2, wherein the pH buffering agent comprises sodium bicarbonate, sodium phosphate, or hydrogen phosphate.

5. The composition according to claim 1, which further comprises a pH adjustment product.

6. The composition according to claim 5, wherein the pH adjustment product comprises a proton pump inhibitor.

7. The composition according to claim 1, which comprises a buffering agent and a pH adjustment product.

8. The composition according to claim 7, which comprises a bicarbonate as a buffering agent and a proton pump inhibitor as a pH adjustment product.

9. The composition according to claim 1, wherein the composition is for oral, enteral, or parenteral administration.

10. The composition according to claim 1, wherein the at least one pancreatic enzyme of animal origin is of mammalian origin.

11. The composition according to claim 10, wherein the at least one pancreatic enzyme is of porcine, bovine, canine, feline, murine, or ovine origin.

12. The composition according to claim 1, wherein the at least one synthetic pancreatic enzyme is produced in a yeast host, a bacterium host, a plant cell host, an insect cell host, or an animal cell host.

13. The composition according to claim 1, wherein the at least one synthetic pancreatic enzyme is synthesized in vitro.

14. The composition according to claim 1, which comprises synthetic lipase, mammalian lipase, mammalian amylase, and mammalian protease.

15. The composition according to claim 1, which is a solid, liquid, or gel.

16. The composition according to claim 1, wherein the single delivery vehicle is selected from a tablet, pill, capsule, sachet, or vial.

17. The composition according to claim 1, wherein the single delivery vehicle comprises a time-controlled tablet, pill, or capsule.

18. The composition according to claim 17, wherein the single delivery vehicle comprises a delayed-release tablet, pill, or capsule.

19. The composition according to claim 1, which comprises lipase, amylase, and protease.

20. The composition according to claim 19, wherein the lipase:amylase:protease ratio is in the range from 1:10:10 to 10:1:1, preferably from 1:1:1 to 1:6:4.

21. The composition according to claim 1, wherein the proportion of synthetic to animal origin pancreatic enzymes ranges from 1% to 99%.

22. A formulation for oral administration in a single delivery vehicle comprising the following enzymes: (i) lipase, (ii) amylase, and (iii) protease, wherein at least one of the enzymes is animal-derived and at least one the enzymes is synthetic.

23. The formulation according to claim 22, wherein the animal-derived enzyme is present in a protein-rich fraction derived from an animal extract.

24. The formulation according to claim 23, wherein the protein-rich fraction comprises isolated lipase, isolated amylase, and/or isolated protease.

25. The formulation according to claim 22, which comprises animal-derived lipase, animal-derived amylase, and animal-derived protease and the animal-derived enzymes are present in the same ratio of lipase:amylase:protease as in the animal extract from which the enzymes have been obtained.

26. The formulation according to claim 22, which comprises animal-derived lipase, animal-derived amylase, and animal-derived protease and the animal-derived enzymes are purified or partially purified to increase the proportion of one or more of the enzymes.

27. The formulation according to claim 22, which comprises synthetic lipase, animal-derived lipase, animal-derived amylase, and animal-derived protease.

28. A method of treating exocrine pancreatic insufficiency (EPI), comprising:
administering via a single delivery vehicle an effective amount of a composition comprising (a) at least one pancreatic enzyme of animal original, and (b) at least one synthetic pancreatic enzyme, to a subject in need thereof.

29. The method according to claim 28, wherein the EPI is a result of or aggravated by cystic fibrosis (CF), chronic pancreatitis (CP), gluten intolerance, alcoholism, or diabetes.

30. A method of manufacturing a composition for the treatment of EPI, the method comprising:
adding at least one synthetic pancreatic enzyme to at least one pancreatic enzyme of animal origin to form a pancreatic enzyme combination; and
filling or forming a single delivery vehicle with the pancreatic enzyme combination.

31. The method according to claim 30, which comprises pre-blending at least one pancreatic enzyme of animal origin and at least one synthetic pancreatic enzyme before filling or forming the delivery vehicle.

32. The method according to claim 30, which comprises forming the pancreatic enzyme combination at the time of creation of the delivery vehicle.

33. A single delivery vehicle for oral administration comprising at least one pancreatic enzyme of animal origin and at least one synthetic pancreatic enzyme, which delivery vehicle is configured to maintain physical separation of the at least one pancreatic enzyme of animal origin and the at least one synthetic pancreatic enzyme until ingested.

34. The single delivery vehicle for oral administration according to claim 33, wherein the at least one pancreatic enzyme of animal origin and the at least one synthetic pancreatic enzyme are present in separate chambers within the single delivery vehicle.

35. The single delivery vehicle according to claim 34, which is configured in a capsule-within-a-capsule format.

36. The single delivery vehicle according to claim 33, wherein the at least one pancreatic enzyme of animal origin is present in a protein-rich fraction derived from an animal extract.

37. The single delivery vehicle according to claim 36, wherein the protein-rich fraction has been further sub-fractionated to contain a single isolated protein.
